(19) United States
(12) Patent Application Publication
(10) Pub. No.: US 2019/0076341 A1
(43) Pub. Date: Mar. 14, 2019

(54) COMPOSITION FOR REDUCTION OF HAIR SPLIT ENDS, USE AND PROCESS THEREOF

(71) Applicant: KAO GERMANY GMBH, Darmstadt, Hessen (DE)

(72) Inventors: Normen LIPINSKI, Darmstadt, Hessen (DE); Frank EBNER, Darmstadt, Hessen (DE)

(73) Assignee: KAO GERMANY GMBH, Darmstadt, Hessen (DE)

(21) Appl. No.: 16/072,271

(22) PCT Filed: Jan. 27, 2016
(86) PCT No.: PCT/EP2016/051639

§ 371 Date: Jul. 24, 2018

(51) Int. Cl. A61K 8/31; A61K 8/81; A61K 8/891; A61K 8/895; A61K 8/58; A61Q 5/00

(52) U.S. Cl. CPC A61K 8/31; A61K 8/8111; A61K 8/8117; A61K 8/891; A61K 8/895; A61K 2800/594; A61Q 5/002; A61K 2800/31; A61K 2800/88; A61K 2800/544; A61K 8/585

(57) ABSTRACT

The present inventions relates to a composition for treating hair, especially human hair, the use of the composition, a process to treat human hair and a kit of parts comprising the composition. The inventors surprisingly found that a composition comprising a silicone vinyl dendrimer, a homopolymeric olefin, monomeric alkanes and/or cyclic silicones, and copolymers comprising olefinic and aromatic monomers mends hair split ends whose effect sustains several washes.

The present invention relates to a composition and process to treat hair split ends, especially human hair. It also relates to the use of the composition to reduce hair split ends. Additionally the invention is on a kit of parts comprising the composition.

Daily styling routines as well as chemical services such as bleaching, coloring and perming may cause serious hair damage, which may finally result in split ends. The products available are neither able to repair/rebind the split hair tips nor show an effect which sustains a hair wash. This requires frequent application of the composition preferably after every hair wash which is time consuming and not economical for the consumers. There is a great need for new products which effectively repair the split ends and/or prevents the formation of split ends.

The present invention starts from the above problems and aims at providing a composition for repairing and/or preventing formation of split ends which lasts at least for several hair washes.

The inventors of the present invention have unexpectedly found that an anhydrous composition comprising a silicone vinyl dendrimer having at least one group reacting via free radical polymerization, one or more alkanes, one or more copolymers consisting of olefin and aromatic monomers and one or more homopolymeric olefins repairs the split ends and the effect lasts for several hair washes. This relieves the consumer from having to cut the hair only for the reason of split ends. Consequently customers can grow their hair to the desired length.

Silicone vinyl dendrimers are known from EP 0963751 (Dow Corning Inc.). EP 1862162 discloses vinylated silicones for the purpose of skin make-up to confer color and transfer resistance. In US 2013/0224130 and Research Disclosure 495005 anhydrous lip stick compositions are disclosed which comprise vinyl silicone dendrimers. However, the publications are silent on the core of the present invention.

Therefore, the first object of the present invention is an anhydrous cosmetic composition comprising:

a) one or more silicone dendrimers with at least one group that reacts via free radical polymerization,
b) One or more monomeric alkanes with a carbon chain length of at least C₁₀ or a siloxane,
c) One or more homopolymeric olefins, and
d) One or more copolymers consisting of olefin and aromatic monomers.

The second object is the use of the anhydrous composition to reduce split ends of hair.

The third object is a process of hair treatment comprising the steps of:

i) Optionally washing the hair with a cleansing composition,
ii) Applying the anhydrous cosmetic composition of the present invention,
iii) Optionally elevating the temperature of the hair ranging from 45° C. to 90° C. for less than 1 min, preferably from 10 to 30 s, wherein the temperature is measured at the surface of the hair fibers,
iv) Optionally rinsing the hair with water.

The fourth object is a kit of parts comprising the anhydrous composition of the present invention.

The vinylated silicone dendrimers according to the present invention possess at least one group that reacts via free radical polymerization. Such groups are acrylic, methacrylic, and vinyl groups. The silicone acrylate dendrimers possess at least one of the groups per dendrimer molecule.

Monoacrlyated dendrimers are available from JNC America under the trade names Silaplane FM 0711, Silaplane FM 0721, Silaplane FM 0725, Silaplane TM 0701, and Silaplane TM 0701T. Bifunctional silicon acrylate dendrimers are also available from JNC America under the trade names Silaplane FM 7711, Silaplane FM 7721, and Silaplane FM 7725.

Vinylated silicon dendrimers according to the present invention are disclosed in EP1055674. Multi-vinylated dendrimers are synthesized by Kim and Hong (Molecules, 2009, Vol. 14, p. 3719-3730).

The most preferred are monoacrylated silicone dendrimers that are sold by Dow Corning under the name DC FA 4002 ID, DC FA 4001 CM, and DC FA 4003 DM.

The total concentration by weight for vinylated silicon dendrimers range from 0.01% to 10%, preferably from 0.1% to 5%, more preferably from 0.15% to 2.5%, most preferably from 0.2% to 2%, by weight calculated to the total of the composition.

Monomeric alkanes with a chain length of at least C₁₀ according to the present invention can be either branched, or linear, saturated, or unsaturated with one or more carbon to carbon double bond, whereas the chain length refers to the total number of carbon atoms within the molecule. Non-limiting examples of such compounds are decane, isodecane, undecane, dodecane, isododecane, tridecane, tetradecane. Particularly preferred are branched alkanes, whereas the most preferred branched alkane is isododecane. The monomeric alkanes are comprised in the composition at a total concentration by weight from 1% to 40%, preferably 1% to 20%, calculated to the total of the composition.

As an alternative to monomeric alkanes, silicones are used according to the present invention. Suitable silicones are cyclic or linear. Suitable cyclic silicones are octamethylcyclotetrasiloxane and decamethylcyclopentasiloxane. The more preferred cyclic silicone is decamethylcyclopentasiloxane. Suitable linear silicones are dimethicone or trimethicone. The more preferred linear silicone is dimethicone. The silicones are comprised in the composition at a total concentration by weight from 0.1% to 40%, preferably 0.1% to 20%, and more preferably 1.0% to 15%, calculated to the total of the composition. It is also possible to use mixtures of monomeric alkanes and cyclic and/or linear silicones. In case of mixtures the total concentration by weight of the mixture is from 1% to 30%, preferably 1% to 20% calculated to the total of the composition whereas the mixing ratio of the compounds may range from 1:100 to 100:1, preferably 50:1 to 1:50, more preferably 20:1 to 1:20 and most preferably 10:1 to 1:10.

The homopolymeric olefin according to the present invention is selected from monomers such as ethene, propene, isopropene, butene, isobutene, pentene, isopentene, hexane, and isohexene. The preferred olefinic homopolymer is polyisobutene. The preferred total concentration by weight of the olefinic homopolymer is from 50% to 90%, calculated to the total of the composition.

Copolymers consisting of olefinic and aromatic monomers according to the present invention comprise olefinic monomers selected from ethylene, propylene, butylene, pentylene, and hexylene and styrene as the aromatic monomer. The copolymer can be copolymerized from one or more of aforementioned olefinic monomers as a starting material with styrene. Furthermore, the composition can comprise one or more of such copolymers. Examples are Ethylene/Propylene/Styrene Copolymer and Butylene/Ethylene/Styrene Copolymer. Copolymers are comprised at a total concentration by weight from 0.01% to 7.5%, preferably from 0.1% to 5%, more preferably from 0.25% to 2.5%, calculated to the total of the composition.

The compositions may further comprise lipophilic ingredients such as vegetable oils, for example, jojoba oil, avocado oil, sunflower seed oil, walnut oil, peanut oil, olive oil, rapeseed oil, cottonseed oil, palm oil, sesame oil, soybean oil, coconut oil, safflower oil, almond oil, macadamia nut oil, grapefruit seed oil, lemon kernel oil, orange kernel oil, apricot kernel oil, castor oil, or any other; liquid paraffins, especially paraffinum perliquidum and paraffinum subliquidum; other silicones such as dimethicones, dimethiconols, aminated silicones with primary, secondary, tertiary or quaternary ammonium groups such as amodimethicone, polysilicone 9, polysilicone 28, and quaternium 80, arylated silicones such as phenyl trimethicone; fatty acid esters such as octyl palmitate, isocetyl palmitate, isopropyl palmitate and octyl stearate, C10- to C36-fatty acid triglycerides, as well as their mixtures. Total concentration of these lipophilic compounds is in the range of 0.1 to 20% by weight, preferably from 1 to 15% by weight, and more preferably from 2 to 10% by weight, calculated to total of the composition.

The composition may comprise one or more ceramide and pseudo-ceramide compound, such as the one according to general formula:

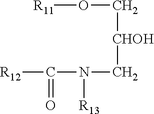

where R11 and R12 are independent from each other alkyl- or. alkenyl group with 10 to 22 carbon atoms, R13 is alkyl or hydroxyl alkyl with 1 to 4 carbon atoms group and n is a number between 1 to 6, preferably 2 or 3. Preferred compound according to the above chemical structure is cetyl-PG-hydroxyethylpalmitamide. Concentration of ceramide type of compounds ranges from 0.01 to 2%, preferably 0.01 to 1% by weight calculated to total the composition.

The composition may comprise antioxidants such as ubiquinone of the formula:

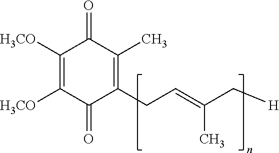

wherein n is a number from 1 to 10. The total concentration of ubiquinone can vary between 0.001% and 10% by weight, calculated to the total of the composition.

The composition may comprise one or more organic solvent such as 2-phenoxyethanol, benzyl alcohol, 2-phenylethanol and 2-benzyloxyethanol. Suitable aliphatic alcohols are ethanol, isopropanol, propanol, n-butanol, isobutanol, t-butanol and 1-pentanol. Total concentration of one or more organic solvent is in the range of 0.1 to 15%, preferably 0.5 to 12.5% and more preferably 1 to 10% and most preferably 1 to 7.5% by weight calculated to the total of each composition.

The anhydrous composition of the present invention may further comprise one or more UV filters which may be selected from oils soluble ones. Non-limiting examples are 4-Aminobenzoic acid and the esters and salts thereof, 2-phenyl benzimidazole-5-sulfonic acid and the alkali and amine salts thereof, 4-dimethyl aminobenzoic acid and the esters and salts thereof, cinnamic acid and the esters and salts thereof, 4-methoxycinnamic acid and the esters and salts thereof, salicylic acid and the esters and salts thereof, 2.4-dihydroxybenzophenone, 2,2′,4,4′-tetra hydroxy-benzophenone, 2-hydroxy-4-methoxybenzophenone and its 5-sulfonic acid or the sodium salt thereof, 2,2′-dihydroxy-4,4′-dimethoxybenzophenone, 2-hydroxy-5-chlorobenzophenone, 2,2′-dihydroxy-4-nnethoxybenzophenone, 2,2′-dihydroxy-4,4′-dimethoxy-5,5′-disulfobenzo-phenone or the sodium salt thereof, 2-hydroxy-4-octyloxybenzophenone, 2-hydroxy-4-methoxy-4′-methylbenzophenone, 3-benzyl-idenecampher, 3-(4′-sulfo)-benzyl-idenebornane-2-one and the salts thereof, 3-(4′-methyl benzylidene)-DL-campher, and/or polysilicone-15. The total UV filter concentration may be in the range of 0.01 to 1% by weight calculated to the total composition.

The composition of the present invention may further comprise oil soluble hair direct dyes. Such hair dyes, for example, are selected from CI 12100, CI 12140, CI 12700, CI 12740, CI 26100, CI 26105, CI 45396, CI 45410, CI 45425, CI 47000, CI 60725, CI 60724, CI 81520, and CI 61565. Hair dye concentration may be in the range from 0.001% to 2.0% calculated to the total of the composition.

The composition may further comprise cosmetically acceptable preservatives and fragrances.

The composition of the present invention may be transparent as analysed by the naked eye in a transparent glass container at a solution thickness of 1 cm or by photometric measurements, and may be turbid measured by aforementioned methods. For the appearance and stability of the composition the miscibility of individual optional compounds needs to be considered by the skilled worker prior to its preparation.

### EXAMPLES

The examples are provided to illustrate the invention, but not to limit it.

### Example 1

Hydrogenated polyisobutene and isododecane were mixed in a vessel until homogeneity was reached. In a next step, the Dow Corning DC FA 4002 ID copolymer was added for the inventive example, then the ethylene/propylene/styrene copolymer was added and the mixture was stirred until homogeneity was reached. In a final step fragrance was added.

| | Inventive % | Comparative % |
|---|---|---|
| Ingredients | by weight | by weight |
| Isododecane | 20.0 | 20.0 |
| DC FA 4002 ID (INCI: isododecane and | 3.5 | — |
| acrylates/polytrimethylsiloxymethacrylate | | |
| copolymer) | | |
| Ethylene/Propylene/Styrene Copolymer | 2.0 | 2.0 |
| Butylene/Ethylene/Styrene Copolymer | 2.0 | 2.0 |
| Fragrance | 0.5 | 0.5 |
| Hydrogenated Polyisobutene | Ad 100.00 | Ad 100.00 |

Split end damaged human hair streaks from Caucasian donors were analysed on their hair split ends extend by light microscopy with a 10× to 20× magnification. 10 hair fibres from each hair streak were analysed. The hair length was between 3 and 5 cm per fibre. The hair was treated with the compositions and the immediate effect was measured by light microscopy and expressed as a ratio of hair with closed ends to the total number of hair fibers by the following equation:

$$Hair split ratio [ \% ] = \frac{Number of hair fibers with closed ends}{Total number of hair fibers} \cdot 100 \%$$

Then the hair was rinsed and shampooed once with Goldwell Dualsenses Scalp Specialist Deep Cleansing Shampoo, analyzed again, and then shampooed for 4 more times. After air-drying the hair, the hair was analyzed by light microscopy and the same calculations from above were performed.

| Split End Recovery Efficacy Test | Inventive | Comparative |
|---|---|---|
| Instant Effect | 82.20% | 20.00% |
| after water rinsing (based on fibers | 85.00% | 0% |
| with an instant effect) | | |
| after shampoo rinsing (based on fibers | 80.00% | 0% |
| with an instant effect) | | |

As a result, hair treated with the inventive composition had a high recovery rate from split ends. Moreover, the effect sustained after rinsing with water and shampooing. Consequently the inventive composition had a beneficial effect superior to the comparative example.

Similar results were observed with the following examples. The preparation of the compositions as well as the effect analysis were performed as outlined above.

### Example 2

| Ingredients | % by weight |
|---|---|
| Cyclopentasiloxane | 20.0 |
| DC FA 4001 CM (INCI: cyclopentasiloxane | 3.5 |
| and acrylates/polytrimethylsiloxymethacrylate | |
| copolymer) | |
| Ethylene/Propylene/Styrene Copolymer | 2.0 |
| Butylene/Ethylene/Styrene Copolymer | 2.0 |
| Fragrance | 0.5 |
| Hydrogenated Polyisobutene | Ad 100.00 |

### Example 3

| Ingredients | % by weight |
|---|---|
| Cyclopentasiloxane | 5.0 |
| Isododecane | 5.0 |
| DC FA 4001 CM | 3.5 |
| Ethylene/Propylene/Styrene Copolymer | 2.0 |
| Butylene/Ethylene/Styrene Copolymer | 2.0 |
| Fragrance | 0.5 |
| Hydrogenated Polyisobutene | Ad 100.00 |

### Example 4

| Ingredients | % by weight |
|---|---|
| Dimethylsiloxane | 5.0 |
| DC FA 4003 DM (INCI: | 3.5 |
| polydimethylsiloxane and acrylates/ | |
| polytrimethylsiloxymethacrylate | |
| copolymer) | |
| Ethylene/Propylene/Styrene Copolymer | 3.0 |
| Butylene/Ethylene/Styrene Copolymer | 3.0 |
| Fragrance | 0.5 |
| Hydrogenated Polyisobutene | Ad 100.00 |

### Example 5

| Ingredients | % by weight |
|---|---|
| Isododecane | 35.0 |
| DC FA 4002 ID | 3.5 |
| Ethylene/Propylene/Styrene Copolymer | 3.0 |
| Butylene/Ethylene/Styrene Copolymer | 3.0 |
| Fragrance | 0.5 |
| Hydrogenated Polyisobutene | Ad 100.00 |

### Example 6

| Ingredients | % by weight |
|---|---|
| Isododecane | 25.0 |
| DC FA 4002 ID | 3.5 |
| Ethylene/Propylene/Styrene Copolymer | 3.0 |
| Butylene/Ethylene/Styrene Copolymer | 3.0 |
| Fragrance | 0.5 |
| Hydrogenated Polyisobutene | Ad 100.00 |

### Examples 7 to 13

| | Example 7 | Example 8 | Example 9 | Example 10 | Example 11 | Example 12 | Example 13 |
|---|---|---|---|---|---|---|---|
| Ingredients | % by weight | % by weight | % by weight | % by weight | % by weight | % by weight | % by weight |
| Isododecane | 20.0 | 20.0 | 20.0 | 20.0 | 20.0 | 20.0 | 20.0 |
| DC FA 4002 ID | 3.5 | 3.5 | 3.5 | 3.5 | 3.5 | 3.5 | 3.5 |
| Ethylene/Propylene/Styrene | 2.0 | 2.0 | 2.0 | 2.0 | 2.0 | 2.0 | 2.0 |
| Copolymer | | | | | | | |
| Butylene/Ethylene/Styrene | 2.0 | 2.0 | 2.0 | 2.0 | 2.0 | 2.0 | 2.0 |
| Copolymer | | | | | | | |
| Fragrance | 0.5 | 0.5 | 0.5 | 0.5 | 0.5 | 0.5 | 0.5 |
| Jojoba oil | 1.0 | 0.2 | — | — | — | — | 1.0 |
| Olive oil | 0.2 | 0.6 | — | — | — | — | — |
| Avocado oil | 0.2 | 0.6 | — | — | — | — | — |
| Ubiquinone | — | — | 2.0 | 2.0 | — | — | — |
| Cetyl-PG- | — | — | — | 0.5 | — | — | 0.5 |
| hydroxyethylpalmitamide | | | | | | | |
| 2-phenoxyethanol | — | — | — | — | 2.5 | 2.5 | — |
| Ethanol | — | — | — | — | 1.0 | 1.0 | — |
| 4-methocinnamic acid | — | — | — | — | — | 0.25 | 0.25 |

| | |
|---|---|
| Hydrogenated | Ad 100.0 |
| polyisobutene | |

1. An anhydrous cosmetic composition comprising:
a) one or more silicone dendrimers with at least one group that reacts via free radical polymerization,
b) One or more monomeric alkanes with a carbon chain length of at least C₁₀ or a siloxane, or mixtures of aforementioned monomeric alkanes with siloxans,
c) One or more homopolymeric olefins, and
d) One or more copolymers consisting of olefin and aromatic monomers.

2. The composition according to claim 1 wherein the one group that reacts via free radical polymerization is selected from acryl, methacryl, or vinyl.

3. The composition according to claim 1 wherein the total concentration by weight of the silicone dendrimer is from 0.01 to 10%, calculated to the total of the composition.

4. The composition according to claim 1 wherein it comprises one or more linear or branched alkanes selected from decane, isodecane, undecane, dodecane, isododecane, tridecane, tetradecane, wherein it is isododecane.

5. The cosmetic composition according to claim 1 wherein the siloxane is cyclic or linear or a mixture of linear and/or cyclic siloxanes.

6. The composition according to claim 1 wherein the component b is comprised at a total concentration in the range of 1% to 40%, by weight calculated to the total of the composition

7. The composition according to claim 1 wherein the one or more homopolymeric olefin monomer(s) is/are selected from ethene, propene, isopropene, butene, isobutene, pentene, isopentene, hexane, and isohexene.

8. The composition according to claim 1, wherein the total concentration by weight of the homopolymeric olefin is from 50% to 90%, calculated to the total of the composition.

9. The composition according to claim 1 wherein the olefin copolymer monomers are selected from ethylene, propylene, butylene, pentylene, and hexylene and the aromatic copolymer units are selected from styrene and pyridine wherein at least one olefinic copolymer unit is polymerized with one or more aromatic unit, preferentially styrene.

10. The composition according to claim 1 wherein the total concentration by weight of the olefinic copolymer is from 0.01% to 7.5%, calculated to the total of the composition.

11. The composition of claim 1 wherein it comprises one or more ingredients selected from fragrance, preservatives, antioxidants, essential oils, vegetable oils, organic solvents, ceramides, ubiquinones, LTV filtering compounds.

12. The composition according to claim 1 wherein it exhibits a viscosity of 1 to 1000 mPas measured with a Brookfield viscosimeter at 25° C.

13. (canceled)

14. (canceled)

15. Kit comprising two or more products chosen from (a), (b), (c) or (d) comprising the composition according to claim 1.
